(19) United States
(12) Patent Application Publication
(10) Pub. No.: US 2026/0232195 A1
(43) Pub. Date: Aug. 13, 2026

(54) WEARABLE QUANTUM SENSOR

(71) Applicant: Toyota Motor Engineering & Manufacturing North America, Inc., Plano, TX (US)

(72) Inventors: Shougo Higashi, Ann Arbor, MI (US); Yukihiro Tadokoro, Ann Arbor, MI (US)

(21) Appl. No.: 18/976,621

(22) Filed: Dec. 11, 2024

(51) Int. Cl. A61B 5/00

(52) U.S. Cl. CPC A61B 5/0059; A61B 5/681; A61B 2562/0285

(57) ABSTRACT

A wearable quantum sensor includes a wearable electronic device, a nanostructured textile attached to the wearable electronic device, and a room temperature spin defect embedded in the nanostructured textile. In some variations, the wearable quantum sensor and/or the wearable electronic device include a light source configured to propagate light onto the room temperature spin defect, a microwave source configured to propagate microwave radiation onto the room temperature spin defect, and a photoluminescent light detector configured to detect and measure a photoluminescent light emitted from the room temperature spin defect.

### TECHNICAL FIELD

The present disclosure generally relates to sensors and particularly to wearable sensors.

### BACKGROUND

Wearable sensors rely on optical technologies to measure or monitor physiological parameters of an individual such as cardiac output, respiration, and blood oxygen levels, among others. And quantum sensors are devices that use properties of quantum mechanics to measure physical properties. For example, quantum sensors utilize spin properties of vacancy centers of materials to measure physical properties of an environment, such as magnetic field strength, electric field strength, temperature, strain, and room temperature.

The present disclosure addresses issues related to wearable sensors, and other issues related to sensors.

### SUMMARY

This section provides a general summary of the disclosure and is not a comprehensive disclosure of its full scope or its features.

In one form of the present disclosure, a wearable quantum sensor includes a wearable electronic device, a nanostructured textile attached to the wearable electronic device, and a room temperature spin defect embedded in the nanostructured textile.

In another form of the present disclosure, a wearable quantum sensor includes a wearable electronic device, a nanostructured textile attached to the wearable electronic device, a room temperature spin defect embedded in the nanostructured textile, a light source and a microwave source configured to propagate light and microwave radiation, respectively, onto the room temperature spin defect.

In still another form of the present disclosure, a wearable quantum sensor includes a wearable electronic device selected from a smart watch, a hearing device, a piece of smart clothing, a wrist band, an arm band, a chest band, a skin patch, a hat, and a mask. The wearable quantum sensor also includes a nanostructured textile attached to the wearable electronic device, a room temperature spin defect embedded in the nanostructured textile, a light source configured to propagate light onto the room temperature spin defect, a microwave source configured to propagate microwave radiation onto the room temperature spin defect, and a photoluminescent light detector configured to detect and measure photoluminescent light emitted from the room temperature spin defect.

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF THE DRAWINGS

The present teachings will become fully understood from the detailed description and the accompanying drawings, wherein:

FIG. 1 is a perspective view of a wearable quantum sensor according to the teachings of the present disclosure;

FIG. 2 is a perspective view of a quantum sensor layer according to the teachings of the present disclosure;

FIG. 3 is an enlarged view of section A-A in FIG. 2;

FIG. 4 illustrates operation of the wearable quantum sensor in FIG. 1; and

FIG. 5 is a graphical plot of photoluminescent intensity as a function of microwave radiation frequency for a room temperature spin defect embedded in a nanostructured textile according to the teachings of the present disclosure;

FIG. 6 is a block diagram of a wearable quantum sensor according to the teachings of the present disclosure; and

FIG. 7 a flow chart for a method of determining a physiological and/or biochemical parameter of an individual wearing a wearable quantum sensor according to the teachings of the present disclosure.

It should be noted that the figures set forth herein are intended to exemplify the general characteristics of the methods and devices among those of the present technology, for the purpose of the description of certain aspects. The figure may not precisely reflect the characteristics of any given aspect and are not necessarily intended to define or limit specific forms or variations within the scope of this technology.

### DETAILED DESCRIPTION

The present disclosure provides a wearable quantum sensor configured to sense and measure various physiological and/or biochemical parameters (also known as “biometric data” or “biometric readings”) of an individual wearing the wearable quantum sensor. The wearable quantum sensor integrates quantum materials into a nanostructured textile and thereby enables the detection of changes in biometric data of the individual. The wearable quantum sensor includes a wearable electronic device, a nanostructured textile, and a room temperature spin defect embedded in the nanostructured textile. The room temperature spin defect is responsive to light and microwave radiation, thereby facilitating the detection and measurement of the biometric data as described in greater detail below.

In some variations, the nanostructured textile includes plasmonic fibers, e.g., silver fibers and/or gold fibers, which amplify the sensitivity of the room temperature spin defect to external stimuli via localized surface plasmon resonance effects. Stated differently, the plasmonic fibers control light by manipulating the interaction between an electromagnetic field and free electrons in the fibers. In this manner, the plasmonic fibers localize light from a light source onto the room temperature spin defect and thereby result in an enhanced signal therefrom.

In some variations, the nanostructured textile includes hollow fibers, such as hollow silver fibers and/or hollow gold fibers. In such variations, the hollow fibers provide additional surface area and facilitate better interaction between the room temperature spin defect and the external environment.

The room temperature spin defect(s) can be a nitrogen vacancy (NV) center in a diamond nanoparticle and/or a negatively charged boron vacancy in hexagonal boron nitride (hBN), among others. In some variations, the room temperature spin defect includes a color center that is utilized to measure physical properties of an environment (e.g., skin, perspiration, etc.) in contact with the wearable quantum sensor. As used herein, the phrase “color center” refers to a crystal defect which introduces or provides additional light absorption or light emission in crystalline materials. In some variations, the color center is an impurity, i.e., a foreign atom. In other variations, the color center is a vacancy.

To activate the room temperature spin defect, the wearable quantum sensor can include a light source configured to propagate light onto to the room temperature spin defect. For example, the light source can be a laser diode, a LED, a vertical-cavity surface-emitting laser, a super-luminescent LED, an organic LED, a quantum dot-based light source, or a fiber optic light source that emits light, among others. It should be understood that the choice of light source depends on the required wavelength, intensity, and power efficiency.

In addition to the light source, the wearable quantum sensor can include a microwave source configured to propagate microwave radiation (signal) onto the room temperature spin defect during optically detected magnetic resonance (ODMR) spectroscopy. The microwave signal causes changes in the spin state of the room temperature spin defect and induces resonance transitions. And resonance transitions modulate the fluorescence (e.g., the wavelength and/or intensity of a photoluminescent light signal) emitted by the room temperature spin defect.

The wearable quantum sensor can also include a photoluminescent light detector configured to detect and measure a photoluminescent light signal emitted from the room temperature spin defect. The detected photoluminescent light signal provides information about the quantum states of the room temperature spin defect, which are correlated with one or more physiological and/or biochemical parameters being monitored.

During operation of the wearable quantum sensor being worn by an individual, light from the light source propagates onto and excites electrons of the room temperature spin defect embedded in the nanostructured textile. A specific wavelength of the light source is chosen to match the absorption characteristics of the room temperature spin defect, ensuring efficient excitation. For instance, NV centers in diamond typically require green light (532 nm) for optimal excitation, while boron vacancies in hBN may require different wavelengths. In other forms, the light source emits longer wavelengths, e.g., 594 nm, 612 nm, 633 nm, 647 nm, 694 nm, among others. In addition, microwave radiation from the microwave source propagates onto and causes changes in the spin state of the room temperature spin defect, thereby inducing resonance transitions. In some variations, the microwave source emits microwave radiation at different frequencies such that the room temperature spin defect emits photoluminescent light signals as a function of the microwave radiation frequencies. Also, the photoluminescent light detector detects the photoluminescent light signals (signals) from the room temperature defect such that the signals are correlated with one or more physiological and/or biochemical parameters being monitored.

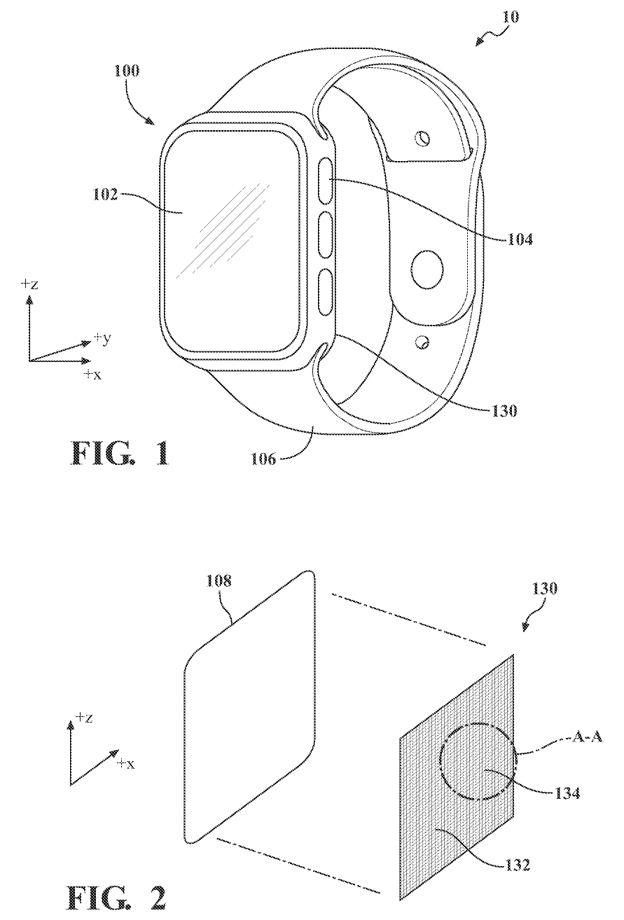

Referring to FIG. 1, a wearable quantum sensor 10 according to the teachings of the present disclosure is shown. And while the wearable quantum sensor 10 is illustrated in the form or as part of a smart watch, it should be understood that the wearable quantum sensor 10 can be in other forms such as hearing device, a piece of smart clothing (i.e., clothing with a quantum sensor embedded therein), a wrist band, an arm band, a chest band, a skin patch, a hat, and a mask, among others.

The wearable quantum sensor 10 includes a wearable electronic device 100 with an attachment device 106, e.g., a band configured to attach the wearable electronic device 100 to an individual. In some variations, the wearable electronic device 100 includes a face 102 with one or more buttons 104 configured to set and/or change operation of the wearable electronic device 100. The wearable quantum sensor 10 also includes a quantum sensor layer 130.

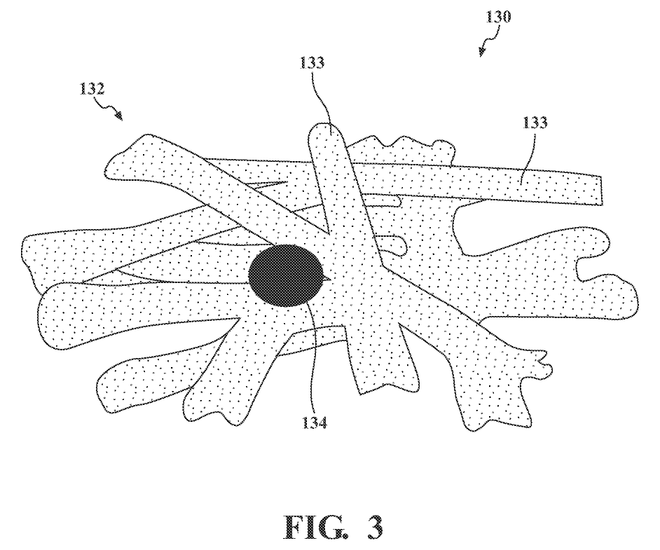

Referring to FIG. 2, an isolated view of the quantum sensor layer 130 is shown. In some variations, and as illustrated in FIG. 2, the quantum sensor layer 130 is adjacent to a back panel 108 (not shown in FIG. 1) of the wearable quantum sensor 10 that is oppositely disposed from the face 102. And in at least one variation the quantum sensor layer 130 is attached to the back panel 108. The quantum sensor layer 130 includes a nanostructured textile 132 and at least one room temperature spin defect 134. And while FIG. 2 illustrates the quantum sensor layer 130 generally being the same size (x-z plane) or substantially covering all of the back panel 108, it should be understood that quantum sensor layers 130 according to the teachings of the present disclosure can be smaller than a panel they are adjacent and/or attached to. That is, in some variations the quantum sensor layer 130 covers between about 1% and about 10% of the back panel 108 (x-z plane), between about 11% and about 20% of the back panel 108, between about 21% and about 30% of the back panel 108, between about 31% and about 40% of the back panel 108, between about 41% and about 50% of the back panel 108, between about 51% and about 60% of the back panel 108, between about 61% and about 70% of the back panel 108, between about 71% and about 80% of the back panel 108, between about 81% and about 90% of the back panel 108, or between about 91% and about 100% of the back panel 108.

Referring to FIG. 3, an enlarged view of section A-A of the quantum sensor layer 130 in FIG. 2 is shown. The quantum sensor layer 130 includes the nanostructured textile 132 and at least one room temperature spin defect 134 (only one room temperature spin defect shown in FIG. 3) embedded in the nanostructured textile 132. The nanostructured textile 132 includes a plurality of fibers 133 that form a matt and/or layer of support for the at least one room temperature spin defect 134 such that the at least one room temperature spin defect 134 has a generally fixed position within and/or on the nanostructured textile 132, on the back panel 108, and/or on the wearable electronic device 100. For example, in some variations a material, e.g., particles, microparticles and/or nanoparticles, that include room temperature spin defects 124 is applied to the nanostructured textile 132 using drop casting, spray coating, or any other method suitable for applying the material to and on the nanostructured textile 132.

Not being bound by theory, the nanostructured textile 132 includes nanofibers treated with nanoparticles, i.e., the plurality of fibers 133 are formed from nanofibers treated with nanoparticles. In some variations, the nanofibers are removed such that a nanoparticle skeleton or shell in the form of elongated hollow fiber shaped structures are formed. For example, in some variations the nanofibers are polymeric fibers formed from a water soluble polymer such as polyvinylpyrrolidone (PVP) and the polymer fibers are removed when exposed to the flow of water. And in such variations, the plurality of fibers 133 are elongated nanoparticle skeletons or shells. And as noted above, in some variations the plurality of fibers 133 are plasmonic fibers that control light by manipulating the interaction between an electromagnetic field and free electrons in the fibers. For example, the plurality of fibers 133 can include gold and/or sliver nanoparticle skeletons or shells such that light from a light source is controlled or directed to one or more room temperature spin defects 134 embedded on and/or within the nanostructured textile 132.

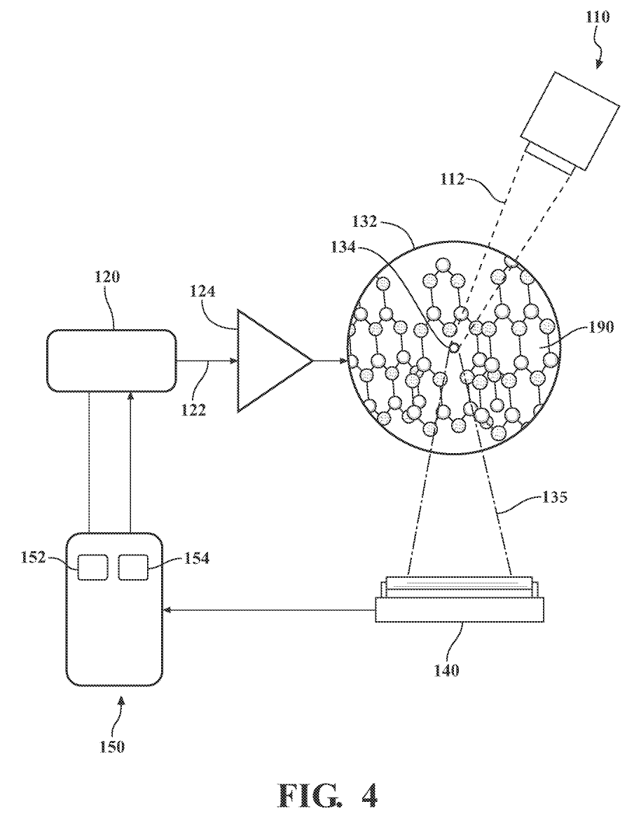

Referring now to FIG. 4, one example of operation of the wearable quantum sensor 10 is illustrated. Particularly, a light source 110 emits a light 112 that contacts or illuminates the room temperature spin defect 134 embedded in the nanostructured textile 132, both of which are in direct contact with skin and/or perspiration 190 of an individual wearing the wearable quantum sensor 10. As used herein, the term “perspiration”, also known as sweat, is defined as the fluid secreted by sweat glands in the skin of mammals. The light 112 excites the electrons of the room temperature spin defect 134 which induces a fluorescence emission 135 therefrom. The light source 110 is, in one or more forms, a 532 nm green laser. In other forms, the light source 110 emits longer laser wavelengths, e.g., 594 nm, 612 nm, 633 nm, 647 nm, 694 nm, among others. For example, the light source 110 may be an indium gallium nitride (InGaN) based laser or InGaN LED light source that emits a 532 nm green laser or a Krypton (Kr) based laser that emits a 647 nm red laser.

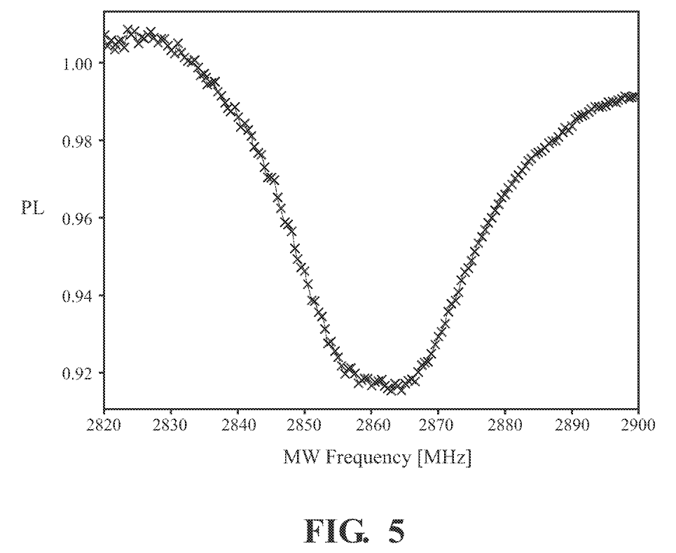

In addition to the light source 110, a microwave source 120 is used to apply a microwave signal 122 to the room temperature spin defect 134, on and /or embedded in the nanostructured textile 132, during optically detected magnetic resonance (ODMR) spectroscopy. The microwave signal is, in one form, amplified by an amplifier 124. The applied microwave signal 122 causes changes in the spin state of the room temperature spin defect 134 and induces resonance transitions. Resonance transitions may modulate the fluorescence (e.g., the wavelength and/or intensity of the fluorescence) emitted by the room temperature spin defect 134. In one or more variations, a photoluminescent light detector 140 detects and analyzes the fluorescence emission 135 emitted by the room temperature spin defect 134. Also, the fluorescence intensity and/or wavelength is measured by the photoluminescent light detector 140 as a function of the microwave signal 122 emitted by the microwave source 120 as illustrated by the graphical plot in FIG. 5.

The photoluminescent light detector data is collected using a microcontroller 150 with a processor 152 and memory 154, and changes in fluorescence as a function of the microwave signal 122 provide insight regarding the skin and/or perspiration 190 of the individual and biometric data associate therewith. For example, in one or more variations, the fluorescence intensity can provide information relating to strength of a magnetic field, an electric field, pH, and/or the temperature of the skin and/or perspiration 190 such that one or more physiological and/or biochemical parameters of the individual can be determined. Non-limiting examples of such physiological and/or biochemical parameters include cardiac output, respiration, and blood oxygen levels.

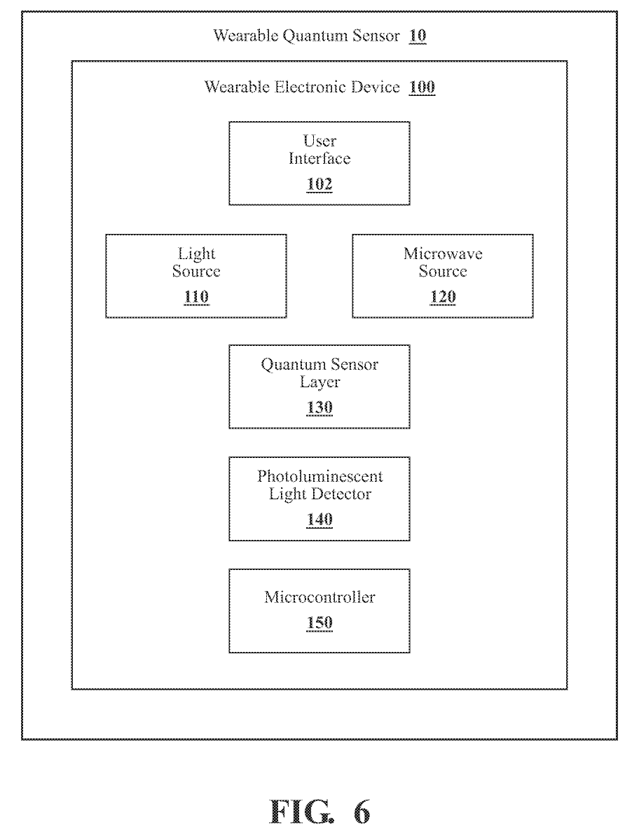

Referring now to FIG. 6, a block diagram of the wearable quantum sensor 10 is shown. The wearable quantum sensor includes the wearable electronic device 100 with a user interface 102 (e.g., the face 102 and/or buttons 104 shown in FIG. 1), the light source 110, the microwave source 120, and the quantum sensor layer 130. The photoluminescent light detector 140 and the microcontroller 150 are also included. And as noted above, the quantum sensor layer 130 can be in direct contact with the skin and/or perspiration of an individual.

Illumination and irradiation of the room temperature spin defect 134 of the quantum sensor layer 130 via the light source 110 and the microwave source 120, respectively, results in a fluorescent signal from the room temperature spin defect 134 that is a function of microwave frequency. For example, in some variations the intensity of the fluorescent signal from the room temperature spin defect 134 varies as a function of microwave frequency (e.g., see FIG. 5). And based on a change or shift of the fluorescent signal (e.g., a shift in the minimum of the curve shown in FIG. 5), biometric information from the individual is provided. For example, a change or shift of the fluorescent signal can provide information on a change in temperature, a change in blood oxygen level, and/or a change in perspiration of the individual wearing the wearable quantum sensor 10.

In some variations, the memory 154 includes instructions that, when executed by the processor 152, cause the processor 152 to compare the fluorescent signal shown in FIG. 5 with a fluorescent signal and/or fluorescent signal data stored in the memory 154. And based on such a comparison, the processor 152 calculates a change in a physiological and/or biochemical parameter of the individual and displays a change in a physiological and/or biochemical parameter of the individual on the face 102. The memory 154 can also include instructions that, when executed by the processor 152, cause the processor 152 to calculate a current physiological and/or biochemical parameter of the individual and/or display a current in a physiological and/or biochemical parameter of the individual on the face 102. It should be understood that the wearable quantum sensor 10 can also wirelessly transmit such physiological and/or biochemical parameter data to a desired remote location.

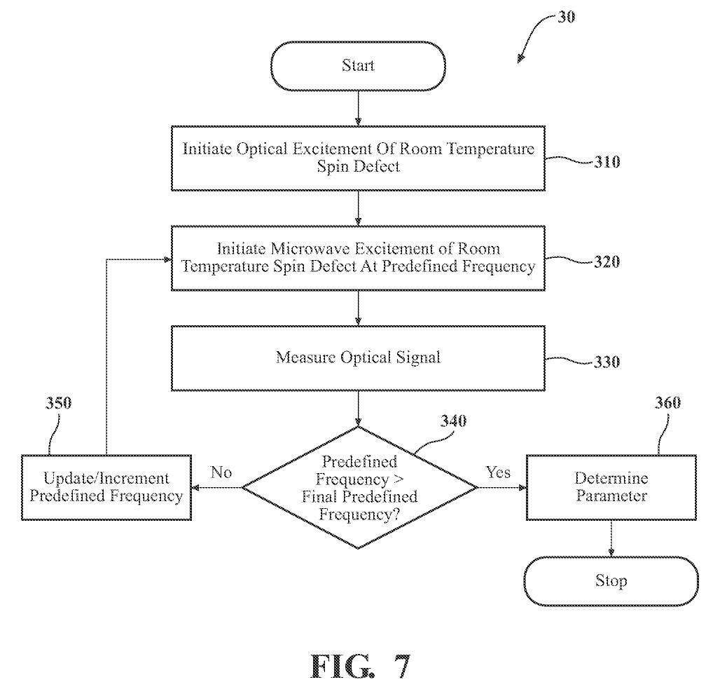

Referring to FIG. 7, and with reference to FIGS. 1-4, a method 30 for determining a physiological and/or biochemical parameter of an individual wearing the wearable quantum sensor 10 is shown. The method 30 includes initiating optical excitement of the room temperature spin defect 134 with the light source 110 at 310. The microwave source 120 initiates microwave excitement of the room temperature spin defect 134 with a predefined frequency at 320, and an optical signal 114 from the optically and microwave excited room temperature spin defect 134 (e.g., the optical signal intensity) is measured with the photoluminescent light detector 140 at 330. The method 30 includes determining if the predefined frequency in step 320 is greater than a final predefined frequency at 340, and if the predefined frequency in step 320 is not greater than the final predefined frequency, the method 30 updates and/or increments the predefined frequency at 350 and returns to 320 where the room temperature spin defect 134 is microwave excited with the updated/increment microwave signal. This cycle, i.e., 320 – 330 – 340 – 350 – 320, continues until the predefined frequency is greater than the predefined final frequency. And when the predefined frequency is greater than the predefined final frequency at 340, the method 30 determines a predefined physiological and/or biochemical parameter of an individual wearing the wearable quantum sensor 10 at 360. In some variations, the microcontroller 150 calculates a value (e.g., using the processor 152) for the predefined physiological and/or biochemical parameter using one or more equations stored in memory 154. In the alternative, or in addition to, the microcontroller 150 determines a value (e.g., using the processor 152) for the predefined physiological and/or biochemical parameter from a look-up table stored in memory 154. In this manner, the wearable quantum sensor 10 and/or method 30 measure an optical signal from the optically and microwave excited room temperature spin defect as a function of a physiological and/or biochemical parameter of an individual wearing the wearable quantum sensor 10.

It should be understood that the wearable quantum sensor offers several advantages over traditional wearable devices. The integration of quantum materials with spin defects into nanostructured textiles allows for a level of precision and accuracy unattainable with conventional optical methods. The incorporation of plasmonic fibers, such as silver or gold, into the textile further amplifies the sensitivity of the quantum sensors through localized surface plasmon resonance effects. Furthermore, the wearable quantum sensors can operate effectively at room temperature, making them suitable for continuous everyday use in wearable devices.

The preceding description is merely illustrative in nature and is in no way intended to limit the disclosure, its application, or uses. Work of the presently named inventors, to the extent it may be described in the background section, as well as forms and/or variations of the description that may not otherwise qualify as prior art at the time of filing, are neither expressly nor impliedly admitted as prior art against the present technology.

As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure, and are not intended to limit the disclosure of the technology or any form or variation thereof. The recitation of multiple forms or variations having stated features is not intended to exclude other forms or variations having additional features, or other forms or variations incorporating different combinations of the stated features.

As used herein the term “about” when related to numerical values herein refers to known commercial and/or experimental measurement variations or tolerances for the referenced quantity. In some variations, such known commercial and/or experimental measurement tolerances are +/- 10% of the measured value, while in other variations such known commercial and/or experimental measurement tolerances are +/- 5% of the measured value, while in still other variations such known commercial and/or experimental measurement tolerances are +/- 2.5% of the measured value. And in at least one variation, such known commercial and/or experimental measurement tolerances are +/- 1% of the measured value.

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The flowcharts and block diagrams in the figures illustrate the architecture, functionality, and operation of possible implementations of systems, methods, and computer program products according to various embodiments. In this regard, a block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The sensors, systems, components, controllers, computers and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a ROM, an EPROM or flash memory, a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain or store a program for use by or in connection with an instruction execution system, apparatus, or device.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one form or variation, or various forms or variations means that a particular feature, structure, or characteristic described in connection with the form or variation or particular system is included in at least one form or variation of the present disclosure. The appearances of the phrase “in one form” or “in one variation” (or variations thereof) are not necessarily referring to the same form or variation. It should be also understood that the various method steps discussed herein do not have to be carried out in the same order as depicted, and not each method step is required in each form or variation.

The foregoing description of the forms and variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

What is claimed is:

1. A wearable quantum sensor comprising:
a wearable electronic device;
a nanostructured textile attached to the wearable electronic device; and
a room temperature spin defect embedded in the nanostructured textile.

2. The wearable quantum sensor according to claim 1, wherein the nanostructured
textile comprises plasmonic fibers selected from the group consisting of silver fibers and gold fibers.

3. The wearable quantum sensor according to claim 2, wherein the nanostructured
textile is formed from the silver fibers.

4. The wearable quantum sensor according to claim 2, wherein the nanostructured
textile is formed from the gold fibers.

5. The wearable quantum sensor according to claim 1, wherein the nanostructured textile comprises hollow fibers selected from the group consisting of hollow silver fibers and hollow gold fibers.

6. The wearable quantum sensor according to claim 5, wherein the nanostructured
textile is formed from the hollow silver fibers.

7. The wearable quantum sensor according to claim 5, wherein the nanostructured
textile is formed from the hollow gold fibers.

8. The wearable quantum sensor according to claim 1, wherein the room temperature spin defect is selected from the group consisting of a nitrogen vacancy center in a diamond nanoparticle and a negatively charged boron vacancy in hexagonal boron nitride.

9. The wearable quantum sensor according to claim 8, wherein the room temperature spin defect is the nitrogen vacancy center in the diamond nanoparticle.

10. The wearable quantum sensor according to claim 8, wherein the room temperature spin defect is the negatively charged boron vacancy in the hexagonal boron nitride.

11. The wearable quantum sensor according to claim 1, further comprising:
a light source configured to propagate light onto the room temperature spin defect;
a microwave source configured to propagate microwave radiation onto the room temperature spin defect; and
a photoluminescent light detector configured to detect and measure a photoluminescent light emitted from the room temperature spin defect.

12. The wearable quantum sensor according to claim 11, wherein the wearable electronic device is selected from the group consisting of a smart watch, a hearing device, a piece of smart clothing, a wrist band, an arm band, a chest band, a skin patch, a hat, and a mask.

13. The wearable quantum sensor according to claim 12, wherein the wearable electronic device is a smart watch.

14. A wearable quantum sensor comprising:
a wearable electronic device;
a nanostructured textile attached to the wearable electronic device;
a room temperature spin defect embedded in the nanostructured textile;
a light source configured to propagate light onto the room temperature spin defect; and
a microwave source configured to propagate microwave radiation onto the room temperature spin defect.

15. The wearable quantum sensor according to claim 14, wherein the nanostructured
textile comprises plasmonic fibers selected from the group consisting of silver fibers and gold fibers.

16. The wearable quantum sensor according to claim 14, wherein the nanostructured textile comprises hollow fibers selected from the group consisting of hollow silver fibers and hollow gold fibers.

17. The wearable quantum sensor according to claim 14, further comprising a photoluminescent light detector configured to detect and measure a photoluminescent light emitted from the room temperature spin defect.

18. The wearable quantum sensor according to claim 14, wherein the wearable electronic device is selected from the group consisting of a smart watch, a hearing device, a piece of smart clothing, a wrist band, an arm band, a chest band, a skin patch, a hat, and a mask.

19. A wearable quantum sensor comprising:
a wearable electronic device selected from the group consisting of a smart watch, a hearing device, a piece of smart clothing, a wrist band, an arm band, a chest band, a skin patch, a hat, and a mask;
a nanostructured textile attached to the wearable electronic device;
a room temperature spin defect embedded in the nanostructured textile;
a light source configured to propagate light onto the room temperature spin defect;
a microwave source configured to propagate microwave radiation onto the room temperature spin defect; and
a photoluminescent light detector configured to detect and measure a photoluminescent light emitted from the room temperature spin defect.

20. The wearable quantum sensor according to claim 19, wherein the nanostructured textile comprises plasmonic fibers selected from the group consisting of silver fibers and gold fibers, and the room temperature spin defect is selected from the group consisting of a nitrogen vacancy center in a diamond nanoparticle- and a negatively charged boron vacancy in hexagonal boron nitride.
